(19) United States
(12) Patent Application Publication
(10) Pub. No.: US 2025/0069749 A1
(43) Pub. Date: Feb. 27, 2025

(54) METHODS AND TECHNIQUES FOR DIAGNOSING INFECTIONS

(71) Applicant: CD DIAGNOSTICS, INC., Claymont, DE (US)

(72) Inventors: Krista Toler, Pierceton, IN (US); Van Thai-Paquette, Quakertown, PA (US); James Parr, Dorking (GB); Pearl Paranjape, Claymont, DE (US)

(21) Appl. No.: 18/813,597

(22) Filed: Aug. 23, 2024

Related U.S. Application Data
(60) Provisional application No. 63/534,229, filed on Aug. 23, 2023.

(51) Int. Cl. G16H 50/20; G16H 50/30

(52) U.S. Cl. CPC G16H 50/20; G16H 50/30

(57) ABSTRACT

A device may A) obtaining plural pieces of training data each of which being an indicator of a periprosthetic joint infection, wherein the plural pieces of training data include at least one combination of data indicating a high probability of a periprosthetic joint infection. A device may B) using the plural pieces of training data to pre-train a machine learning model. A device may C) wherein the plural pieces of training data of B) correspond to a combination of data that is associated with periprosthetic joint infection. A device may D) feeding plural pieces of data obtained from a subject to the machine learning model. A device may E) determining the likelihood of a subject having a periprosthetic joint infection based on an output of the machine learning model.

### CROSS REFERENCE TO RELATED APPLICATION

This application claims the benefit of priority to U.S. provisional patent application No. 63/534,229, which was filed on Aug. 23, 2023, titled “METHODS AND TECHNIQUES FOR DIAGNOSING INFECTIONS”, and is incorporated herein by reference in its entirety.

### BACKGROUND

The numbers of primary total hip and knee arthroplasties have been increasing over time. With the increase in prosthetic joint implantations, serious complications of periprosthetic joint infections (PJI) of the hip and knee is also on the rise. It is therefore desirable to develop methods and techniques for diagnosing periprosthetic joint infections.

### SUMMARY OF THE INVENTION

In some aspects, the techniques described herein relate to a method including steps of: A) obtaining plural pieces of training data each of which being an indicator of a periprosthetic joint infection, wherein the plural pieces of training data include at least one combination of data indicating a high probability of a periprosthetic joint infection; B) using the plural pieces of training data to pre-train a machine learning model; C) wherein the plural pieces of training data of B) correspond to a combination of data that is associated with periprosthetic joint infection; D) feeding plural pieces of data obtained from a subject to the machine learning model E) determining the likelihood of a subject having a periprosthetic joint infection based on an output of the machine learning model.

In some aspects, the techniques described herein relate to a method including steps of: A) obtaining plural pieces of training data each of which being an indicator of a periprosthetic joint infection, wherein the plural pieces of training data include at least one combination of data indicating a high probability of a periprosthetic joint infection; B) using the plural pieces of training data to pre-train a machine learning model; C) wherein the plural pieces of training data of B) correspond to a combination of data that is associated with periprosthetic joint infection; D) feeding plural pieces of data obtained from a subject to the machine learning model; E) determining the likelihood of a subject having a periprosthetic joint infection based on an output of the machine learning model; and F) treating the periprosthetic joint infection.

### BRIEF DESCRIPTION OF THE DRAWING

FIG. 1 is a block diagram of an example of an environment including a system for neural network training.

FIG. 2 illustrates, by way of example, a block diagram of an embodiment of a machine in the example form of a computer system within which instructions, for causing the machine to perform any one or more of the methodologies discussed herein, may be executed.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

Infections such as periprosthetic joint infection (PJI) can be difficult to diagnose, and an accurate diagnosis is vitally important for making proper clinical care decisions for patients. Current clinical guidelines recommend using multi-biomarker algorithms to assign points based on an independent clinical cutoff for each biomarker. These guidelines are challenging to implement, do not leverage the full power of continuous data, and result in an “inconclusive” category that confuses preoperative decision-making.

Currently, there is no single definition for the diagnosis of periprosthetic joint infection that's been accepted as the reference standard. The most recent published diagnostic definition in the United States is the 2018 International Consensus Meeting (ICM) criteria, which only garnered 68% agreement among the delegates. In addition, diagnosis based on the current accepted criteria (2018 MSIS, 2018 ICM, 2021 EBJIS) results in 5-12% of patients falling into the “Inconclusive” category, making actionable interpretation difficult for this cohort. Furthermore, biomarker results required as inputs for these criteria come from multiple diagnostic platforms (serum, imaging, body fluid, etc.), which makes full utilization difficult for patients with an incomplete panel of test results.

There is accordingly a need to develop a more robust and accurate protocol for diagnosing infections such as periprosthetic joint infection. Although diagnosing periprosthetic joint infection is extensively discussed herein, it is within the scope of this disclosure to use the methods to diagnose other conditions such as septic arthritis, osteoarthritis, or any other arthrosis. It is understood that any reference to periprosthetic joint infection can apply equally to the aforementioned other infections and/or inflammatory state(s).

The diagnostic method generally includes obtaining plural pieces of training data. Each piece of data is an indicator of an infection such as periprosthetic joint infection. The indicators can be obtained by a test measuring protein concentration and/or total protein content, a test measuring red blood cell concentration, a test measuring white blood cell concentration, a neutrophil or polymorphonuclear cell percentage, a test measuring c-reactive protein concentration, a test measuring alpha defensin concentration, a test to detect presence of microbial antigen, a test measuring calprotectin, a test measuring neutrophil elastase, a test measuring leukocyte esterase, a test measuring lipocalcin, a test measuring monocyte-to-lymphocyte ratio, a test measuring neutrophil-to-lymphocyte ratio, a test measuring platelet-to-lymphocyte ratio, a test measuring absolute neutrophil count, a test measuring d-dimer, a test measuring erythrocyte sedimentation rate, a test measuring lactate or L-lactate concentration, a test for crystal identification, a subject's age, the affected joint, and a subject's gender. The plural number can be as few as two but can be any other plural number as needed. In some aspects, the plural pieces of training data include at least one combination of data that is thought to indicate a high probability of a periprosthetic joint infection.

The diagnostic method described herein leverages machine learning techniques to improve the accuracy and reliability of periprosthetic joint infection diagnosis. By utilizing a combination of clinical, laboratory, and patient-specific data, the method aims to overcome the limitations of current diagnostic criteria and reduce the number of inconclusive results

The plural pieces of training data are used to pre-train a machine learning model. That is, the plural pieces of training data are chosen from those that correspond to a combination of data that is associated with periprosthetic joint infection. Over number of runs, the model is able to associate data combination with periprosthetic joint infection. Conversely, a plurality of data that is not associated with periprosthetic joint infection can be fed into the model to train the machine on what is not a combination to associate with periprosthetic joint infection. The plural pieces of data are obtained from multiple subjects as opposed to a singular subject.

To determine the likelihood of an individual subject having periprosthetic joint infection, a plurality of data is obtained from an individual subject. Each piece of data is a potential indicator of an infection such as periprosthetic joint infection. The indicators can be obtained by a test measuring protein concentration and/or total protein content, a test measuring red blood cell concentration, a test measuring white blood cell concentration, a neutrophil or polymorphonuclear cell percentage, a test measuring c-reactive protein concentration, a test measuring alpha defensin concentration, a test to detect presence of microbial antigen, a test measuring calprotectin, a test measuring neutrophil elastase, a test measuring leukocyte esterase, a test measuring lipocalcin, a test measuring monocyte-to-lymphocyte ratio, a test measuring neutrophil-to-lymphocyte ratio, a test measuring platelet-to-lymphocyte ratio, a test measuring absolute neutrophil count, a test measuring d-dimer, a test measuring erythrocyte sedimentation rate, a test measuring lactate or L-lactate concentration, a test for crystal identification, a subject's age, the affected joint, and a subject's gender. The plural number can be as few as two but can be any other plural number as needed. In some aspects, the plural pieces of training data include at least one combination of data that is thought to indicate a high probability of a periprosthetic joint infection.

The machine learning model used in this method can be trained on large datasets of historical patient information, including both confirmed cases of periprosthetic joint infection and non-infected controls. This allows the model to learn complex patterns and relationships between various indicators that may not be apparent through traditional statistical analysis. The use of a machine learning model in this diagnostic method offers several advantages over traditional diagnostic criteria. Firstly, it can handle missing data more effectively, allowing for diagnosis even when certain test results are unavailable. Secondly, it can assign different weights to various indicators based on their predictive power, rather than treating all indicators equally. Lastly, it can provide a continuous probability score rather than a binary classification, allowing for more nuanced clinical decision-making.

The specific combination that is the plurality of data is generally understood to constitute a panel. The composition of the panel can be predetermined or ordered by a healthcare professional. Alternatively, the healthcare professional obtaining the panel can decide which plurality of data to include on the panel. The plurality of data does not have to be obtained by a single healthcare professional. The plurality of data indeed can be collected from a plurality of different healthcare professionals that are trained or available to collect specific types of data. The plurality of data used for training or obtained from the subject can be obtained from synovial fluid, blood, serum, wound exudate, urine, saliva, sebum, tissue swab, or a mixture thereof.

The machine learning model used to assess the likelihood of the subject having periprosthetic joint infection is the same machine learning model that training data is used on. Examples of suitable machine learning models include a logistic regression model, a support vector machine model, a decision trees model, a random forests model, an adaptive boosting trees model, a gradient boosting trees model, an explainable boosting machine model, a nearest neighbors model, a neural networks model, a KMeans model, a gaussian mixture model, a hierarchical clustering model, a density-based spatial clustering of applications with noise (DBSCAN) model, a fuzzy clustering model, a principal component analysis (PCA) model, a linear discriminant analysis (LDA) model, a factor analysis of mixed data or factorial analysis of mixed data (FAMD) model, a single value decomposition (SVD) model, and a t-distributed stochastic neighbor embedding (t-SNE) model.

A logistic regression model itself models probability of output in terms of input and does not perform statistical classification. But the model can be used to make a classifier, for instance by choosing a cutoff value and classifying inputs with probability greater than the cutoff as one class, below the cutoff as the other; this is a common way to make a binary classifier.

A support vector machine can be trained via examples that are marked as belonging to one of two categories (e.g., likely association with periprosthetic joint infection and unlikely association with periprosthetic joint infection). A support vector machine training algorithm builds a model that assigns new examples to one category or the other, making it a non-probabilistic binary linear classifier (although methods such as Platt scaling exist to use support vector machine in a probabilistic classification setting). Support vector machine maps training examples to points in space so as to maximize the width of the gap between the two categories. New examples are then mapped into that same space and predicted to belong to a category based on which side of the gap they fall.

A decision trees model is a flowchart-like structure in which each internal node represents a “test” on an attribute (e.g. whether a biomarker result is above or below a certain threshold), each branch represents the outcome of the test, and each terminal leaf node represents a class label (decision taken after computing all attributes). The paths from root to leaf represent classification rules. In decision analysis, a decision tree and the closely related influence diagram are used as a visual and analytical decision support tool, where the expected values (or expected utility) of competing alternatives are calculated.

Random forests or random decision forests is an ensemble learning method for classification, regression and other tasks that operates by constructing a multitude of decision trees at training time. For classification tasks, the output of the random forest is the class selected by most trees. For regression tasks, the mean or average prediction of the individual trees is returned. Random decision forests address the common issues of overfitting to their training set that is often observed in Decision Trees algorithms.

A neural network is a computational model inspired by the structure of the human brain. It comprises interconnected nodes, or “neurons,” organized in layers. Information flows through these layers, with each neuron performing calculations on incoming data and passing the results to the next layer. Neural networks are commonly used for various tasks, such as pattern recognition, classification, regression, and complex data processing. They are trained on large datasets to learn relationships and patterns in the data, adjusting the connections (weights) between neurons to improve their performance over time.

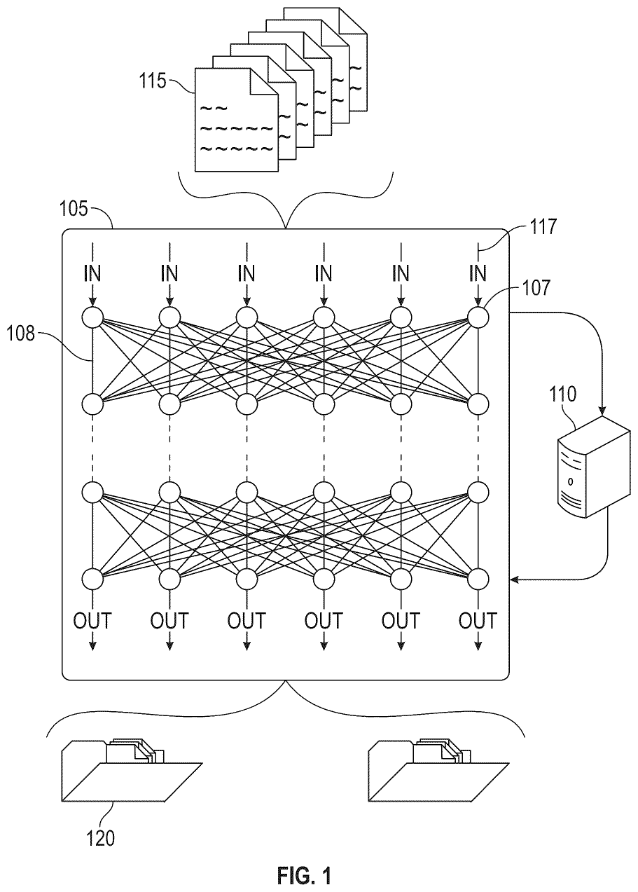

FIG. 1 is a block diagram of an example of an environment including a system for neural network training. The analysis operation 128, GNN 104, decoder 330, encoder 430, decoder 426, or the like can include an NN that can be trained in accord with FIG. 1. The resulting NN can predict entity interactions using interacting dynamic graphs. The system includes an artificial NN (ANN) 605 that is trained using a processing node 610. The processing node 610 may be a central processing unit (CPU), graphics processing unit (GPU), field programmable gate array (FPGA), digital signal processor (DSP), application specific integrated circuit (ASIC), or other processing circuitry. In an example, multiple processing nodes may be employed to train different layers of the ANN 605, or even different nodes 607 within layers. Thus, a set of processing nodes 610 is arranged to perform the training of the ANN 605.

The set of processing nodes 610 is arranged to receive a training set 615 for the ANN 605. The ANN 605 comprises a set of nodes 607 arranged in layers (illustrated as rows of nodes 607) and a set of inter-node weights 608 (e.g., parameters) between nodes in the set of nodes. In an example, the training set 615 is a subset of a complete training set. Here, the subset may enable processing nodes with limited storage resources to participate in training the ANN 605.

The training data may include multiple numerical values representative of a domain, such as a word, symbol, other part of speech, or the like. Each value of the training or input 617 to be classified after ANN 605 is trained, is provided to a corresponding node 607 in the first layer or input layer of ANN 605. The values propagate through the layers and are changed by the objective function.

As noted, the set of processing nodes is arranged to train the neural network to create a trained neural network. After the ANN is trained, data input into the ANN will produce valid classifications 620 (e.g., the input data 617 will be assigned into categories), for example. The training performed by the set of processing nodes 607 is iterative. In an example, each iteration of the training the ANN 605 is performed independently between layers of the ANN 605. Thus, two distinct layers may be processed in parallel by different members of the set of processing nodes. In an example, different layers of the ANN 605 are trained on different hardware. The members of different members of the set of processing nodes may be located in different packages, housings, computers, cloud-based resources, etc. In an example, each iteration of the training is performed independently between nodes in the set of nodes. This example is an additional parallelization whereby individual nodes 607 (e.g., neurons) are trained independently. In an example, the nodes are trained on different hardware. The neural network model, in particular, can be designed with multiple hidden layers to capture complex non-linear relationships between the input data. This architecture allows the model to learn hierarchical features from the raw input data, potentially uncovering novel biomarkers or combinations of indicators that are highly predictive of periprosthetic joint infection.

Artificial Neural Networks (ANNs) are a fundamental type of neural network. They consist of an input layer, one or more hidden layers, and an output layer. Neurons in each layer are connected to neurons in subsequent layers through weighted connections. ANNs are capable of learning complex nonlinear relationships in data and are widely used in tasks like image and speech recognition, natural language processing, and more.

Convolutional Neural Networks (CNNs) are a specialized type of neural network designed for processing grid-like data, such as images and videos. CNNs employ convolutional layers that apply filters to input data, allowing them to automatically learn and identify hierarchical patterns and features in visual data. This makes CNNs highly effective for tasks like image classification, object detection, and image generation. A fuzzy logic model is a form of many-valued logic in which the truth value of variables may be any real number between 0 and 1. It is employed to handle the concept of partial truth, where the truth value may range between completely true and completely false. By contrast, in Boolean logic, the truth values of variables may only be the integer values 0 or 1.

The plurality of data obtained from the subject can be fed into one machine learning model. Alternately, the same plurality of data or a subset of the plurality of data can be fed into a second machine learning model that is different from the initial machine learning model. It is additionally possible for the plurality of data to be fed into a third machine learning model, fourth machine learning model, and so forth. The ability to subject the same plurality of data to different machine learning models can increase the accuracy of the periprosthetic joint infection diagnosis.

Ultimately, the machine learning model or models used produce an output to aid in determining the likelihood of a subject having a periprosthetic joint infection. The output can take many different forms such as a threshold value, a probability score, a rank-percentile, a confidence score, a categorical range, or a combination thereof. If the output is a threshold value, it can be compared against a known value to determine whether a periprosthetic joint infection is likely or unlikely. The specific threshold value may include a rule-in cutoff threshold value or a rule-out cutoff threshold value, or both. It's important to note that while this machine learning-based diagnostic method can significantly improve the accuracy of periprosthetic joint infection diagnosis, it should be used in conjunction with clinical judgment. The output of the model should be interpreted in the context of the patient's overall clinical picture, including physical examination findings and the patient's medical history.

In the event that a subject is determined to have periprosthetic joint infection, a healthcare professional can proceed to treat the periprosthetic joint infection. Treatments can include using a combination of powerful antibiotics as well as draining the infected synovial fluid. It's likely that antibiotics will need to be administered immediately to avoid the spread of the infection. Intravenous (IV) antibiotics are given, usually requiring admission to the hospital for initial treatment. The treatment, however, may be continued on an outpatient basis at home with the assistance of a home health nursing service.

In some aspects of the disclosure, it is helpful to use the disclosed method to determine the likelihood of periprosthetic joint infection in a subject who had been previously tested for a periprosthetic joint infection and received an inconclusive result or negative result.

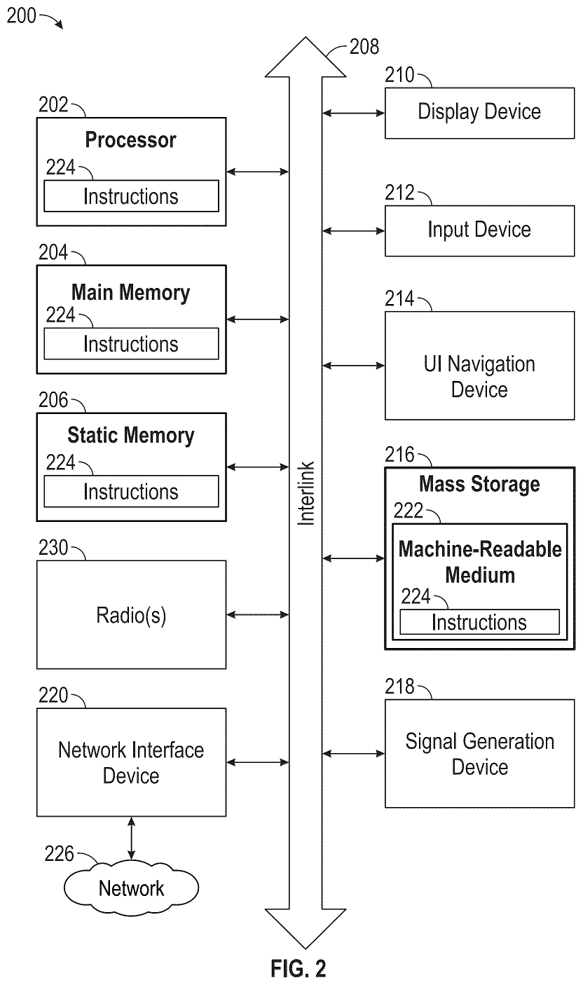

FIG. 2 illustrates, by way of example, a block diagram of an embodiment of a machine in the example form of a computer system 200 within which instructions, for causing the machine to perform any one or more of the methodologies discussed herein, may be executed. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 200 includes a processor 202 (e.g., a central processing unit (CPU), a graphics processing unit (GPU) or both), a main memory 204 and a static memory 206, which communicate with each other via a bus 208. The computer system 200 may further include a video display unit 210 (e.g., a liquid crystal display (LCD) or a cathode ray tube (CRT)). The computer system 200 also includes an alphanumeric input device 212 (e.g., a keyboard), a user interface (UI) navigation device 214 (e.g., a mouse), a mass storage unit 216, a signal generation device 218 (e.g., a speaker), a network interface device 220, and a radio 230 such as Bluetooth, WWAN, WLAN, and NFC, permitting the application of security controls on such protocols.

The mass storage unit 216 includes a machine-readable medium 222 on which is stored one or more sets of instructions and data structures (e.g., software) 224 embodying or utilized by any one or more of the methodologies or functions described herein. The instructions 224 may also reside, completely or at least partially, within the main memory 204 and/or within the processor 202 during execution thereof by the computer system 200, the main memory 204 and the processor 202 also constituting machine-readable media.

While the machine-readable medium 222 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding, or carrying instructions for execution by the machine and that cause the machine to perform any one or more of the methodologies of the present invention, or that is capable of storing, encoding, or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), Electrically Erasable Programmable Read-Only Memory (EEPROM), and flash memory devices; magnetic disks such as internal hard disks and removable disks; magneto-optical disks; and CD-ROM and DVD-ROM disks.

The instructions 224 may further be transmitted or received over a communications network 226 using a transmission medium. The instructions 224 may be transmitted using the network interface device 220 and any one of a number of well-known transfer protocols (e.g., HTTP). Examples of communication networks include a local area network (“LAN”), a wide area network (“WAN”), the Internet, mobile telephone networks, Plain Old Telephone (POTS) networks, and wireless data networks (e.g., WiFi and WiMax networks). The term “transmission medium” shall be taken to include any intangible medium that is capable of storing, encoding, or carrying instructions for execution by the machine, and includes digital or analog communications signals or other intangible media to facilitate communication of such software.

### Exemplary Aspects.

The following exemplary aspects are provided, the numbering of which is not to be construed as designating levels of importance:

Aspect 1 provides a method comprising steps of:
A) obtaining plural pieces of training data each of which being an indicator of a periprosthetic joint infection, wherein the plural pieces of training data include at least one combination of data indicating a high probability of a periprosthetic joint infection;
B) using the plural pieces of training data to pre-train a machine learning model;
C) wherein the plural pieces of training data of B) correspond to a combination of data that is associated with periprosthetic joint infection;
D) feeding plural pieces of data obtained from a subject to the machine learning model
E) determining the likelihood of a subject having a periprosthetic joint infection based on an output of the machine learning model.

Aspect 2 provides the method of Aspect 1, further comprising F) treating the periprosthetic joint infection.

Aspect 3 provides the method of Aspect 2, wherein treating the periprosthetic joint infection comprises treating the subject with an antibiotic.

Aspect 4 provides the method of any of Aspects 1-3, wherein, the plurality of data of A) and D) includes at least two of a test measuring protein concentration and/or total protein content, a test measuring red blood cell concentration, a test measuring white blood cell concentration, a neutrophil or polymorphonuclear cell percentage, a test measuring c-reactive protein concentration, a test measuring alpha defensin concentration, a test to detect presence of microbial antigen, a test measuring calprotectin, a test measuring neutrophil elastase, a test measuring leukocyte esterase, a test measuring lipocalcin, a test measuring monocyte-to-lymphocyte ratio, a test measuring neutrophil-to-lymphocyte ratio, a test measuring platelet-to-lymphocyte ratio, a test measuring absolute neutrophil count, a test measuring d-dimer, a test measuring erythrocyte sedimentation rate, a test measuring lactate or L-lactate, a test for crystal identification, the affected joint, a subject's age, and a subject's gender.

Aspect 5 provides the method of any of Aspects 1-4, wherein the plurality of data comprises a panel of information that is fed into the machine learning model.

Aspect 6 provides the method of any of Aspects 1-5, wherein the machine learning model comprises at least one of a logistic regression model, a support vector machine model, a decision trees model, a random forests model, an adaptive boosting trees model, a gradient boosting trees model, an explainable boosting machine model, a nearest neighbors model, a neural networks model, a KMeans model, a gaussian mixture model, a hierarchical clustering model, a density-based spatial clustering of applications with noise (DBSCAN) model, a fuzzy clustering model, a principal component analysis (PCA) model, a linear discriminant analysis (LDA) model, a factor analysis of mixed data or factorial analysis of mixed data (FAMD) model, a single value decomposition (SVD) model, and a t-distributed stochastic neighbor embedding (t-SNE) model.

Aspect 7 provides the method of any of Aspects 1-6, wherein the machine learning model is a first machine learning model and the method further comprises subjecting the plurality of data to a second machine learning model that is different from the first machine learning model.

Aspect 8 provides the method of any of Aspects 1-7, wherein the output of the machine learning tool is compared against a threshold value, the output of the machine learning tool is delivered as a probability score, the output of the machine learning tool is delivered as a rank-percentile, the output of the machine learning tool is delivered as a confidence score, the output of the machine learning tool is delivered as a categorical range, or a combination thereof.

Aspect 9 provides the method of Aspect 8, further comprising G) determining the threshold value.

Aspect 10 provides the method of any of Aspects 1-9, wherein the plural pieces of data are obtained by one or more health care providers.

Aspect 11 provides the method of any of Aspects 1-10, wherein the plural pieces of data at A) is obtained from multiple subjects.

Aspect 12 provides the method of any of Aspects 1-11, wherein the subject had been previously tested for a periprosthetic joint infection and received an inconclusive result.

Aspect 13 provides a method comprising steps of:
A) obtaining plural pieces of training data each of which being an indicator of a periprosthetic joint infection, wherein the plural pieces of training data include at least one combination of data indicating a high probability of a periprosthetic joint infection;
B) using the plural pieces of training data to pre-train a machine learning model;
C) wherein the plural pieces of training data of B) correspond to a combination of data that is associated with periprosthetic joint infection;
D) feeding plural pieces of data obtained from a subject to the machine learning model;
E) determining the likelihood of a subject having a periprosthetic joint infection based on an output of the machine learning model; and
F) treating the periprosthetic joint infection.

Aspect 14 provides the method of Aspect 13, wherein treating the periprosthetic joint infection comprises treating the subject with an antibiotic.

Aspect 15 provides the method of any of Aspects 1 or 14, wherein, the plurality of data of A) and D) includes at least two of a test measuring protein concentration and/or total protein content, a test measuring red blood cell concentration, a test measuring white blood cell concentration, a neutrophil or polymorphonuclear cell percentage, a test measuring c-reactive protein concentration, a test measuring alpha defensin concentration, a test to detect presence of microbial antigen, a test measuring calprotectin, a test measuring neutrophil elastase, a test measuring leukocyte esterase, a test measuring lipocalcin, a test measuring monocyte-to-lymphocyte ratio, a test measuring neutrophil-to-lymphocyte ratio, a test measuring platelet-to-lymphocyte ratio, a test measuring absolute neutrophil count, a test measuring d-dimer, a test measuring erythrocyte sedimentation rate, a test measuring lactate or L-lactate, the affected joint, a test for crystal identification, a subject's age, and a subject's gender.

Aspect 16 provides the method of any of Aspects 1-15, wherein the plurality of data comprises a panel of information that is fed into the machine learning model.

Aspect 17 provides the method of any of Aspects 1-16, wherein the machine learning model comprises at least one of a logistic regression model, a support vector machine model, a decision trees random forests model, an adaptive boosting trees model, a gradient boosting trees model, an explainable boosting machine model, a nearest neighbors model, a neural networks model, a KMeans model, a gaussian mixture model, a hierarchical clustering model, a density-based spatial clustering of applications with noise (DBSCAN) model, a fuzzy clustering model, a principal component analysis (PCA) model, a linear discriminant analysis (LDA) model, a factor analysis of mixed data or factorial analysis of mixed data (FAMD) model, a single value decomposition (SVD) model, and a t-distributed stochastic neighbor embedding (t-SNE) model.

Aspect 18 provides the method of any of Aspects 1-17, wherein the machine learning model is a first machine learning model and the method further comprises subjecting the plurality of data to a second machine learning model that is different from the first machine learning model.

Aspect 19 provides the method of any of Aspects 1-18, wherein the output of the machine learning tool is compared against a threshold value, the output of the machine learning tool is delivered as a probability score, the output of the machine learning tool is delivered as a rank-percentile, the output of the machine learning tool is delivered as a confidence score, the output of the machine learning tool is delivered as a categorical range, or a combination thereof.

Aspect 20 provides the method of Aspect 19, further comprising G) determining the threshold value.

Aspect 21 provides the method of any of Aspects 1-20, wherein the plural pieces of data are obtained by one more health care providers.

Aspect 22 provides the method of any of Aspects 1-21, wherein the plural pieces of data at A) is obtained from multiple subjects.

Aspect 23 provides the method of any of Aspects 1-22, wherein the subject had been previously tested for a periprosthetic joint infection and received an inconclusive result.

What is claimed is:

1. A method comprising steps of:
A) obtaining plural pieces of training data each of which being an indicator of a periprosthetic joint infection, wherein the plural pieces of training data include at least one combination of data indicating a high probability of a periprosthetic joint infection;
B) using the plural pieces of training data to pre-train a machine learning model;
C) wherein the plural pieces of training data of B) correspond to a combination of data that is associated with periprosthetic joint infection;
D) feeding plural pieces of data obtained from a subject to the machine learning model;
E) determining the likelihood of a subject having a periprosthetic joint infection based on an output of the machine learning model.

2. The method of claim 1, further comprising F) treating the periprosthetic joint infection.

3. The method of claim 2, wherein treating the periprosthetic joint infection comprises treating the subject with an antibiotic.

4. The method of claim 1, wherein, the plurality of data of A) and D) includes at least two of a test measuring protein concentration and/or total protein content, a test measuring red blood cell concentration, a test measuring white blood cell concentration, a neutrophil or polymorphonuclear cell percentage, a test measuring c-reactive protein concentration, a test measuring alpha defensin concentration, a test to detect presence of microbial antigen, a test measuring calprotectin, a test measuring neutrophil elastase, a test measuring leukocyte esterase, a test measuring lipocalcin, a test measuring monocyte-to-lymphocyte ratio, a test measuring neutrophil-to-lymphocyte ratio, a test measuring platelet-to-lymphocyte ratio, a test measuring absolute neutrophil count, a test measuring d-dimer, a test measuring erythrocyte sedimentation rate, a test measuring lactate or L-lactate, the affected joint, a subject's age, and a subject's gender.

5. The method of claim 1, wherein the plurality of data comprises a panel of information that is fed into the machine learning model.

6. The method of claim 1, wherein the machine learning model comprises at least one of a logistic regression model, a support vector machine model, a decision trees model, a random forests model, an adaptive boosting trees model, a gradient boosting trees model, an explainable boosting machine model, a nearest neighbors model, a neural networks model, a KMeans model, a gaussian mixture model, a hierarchical clustering model, a density-based spatial clustering of applications with noise (DBSCAN) model, a fuzzy clustering model, a principal component analysis (PCA) model, a linear discriminant analysis (LDA) model, a factor analysis of mixed data or factorial analysis of mixed data (FAMD) model, a single value decomposition (SVD) model, and a t-distributed stochastic neighbor embedding (t-SNE) model.

7. The method of claim 1, wherein the machine learning model is a first machine learning model and the method further comprises subjecting the plurality of data to a second machine learning model that is different from the first machine learning model.

8. The method of claim 1, wherein the output of the machine learning tool is compared against a threshold value, the output of the machine learning tool is delivered as a probability score, the output of the machine learning tool is delivered as a rank-percentile, the output of the machine learning tool is delivered as a confidence score, the output of the machine learning tool is delivered as a categorical range, or a combination thereof.

9. The method of claim 8, further comprising G) determining the threshold value.

10. The method of claim 1, wherein the plural pieces of data are obtained by one or more health care providers.

11. The method of claim 1, wherein the plural pieces of data at A) is obtained from multiple subjects.

12. The method of claim 1, wherein the subject had been previously tested for a periprosthetic joint infection and received an inconclusive result.

13. A method comprising steps of:
A) obtaining plural pieces of training data each of which being an indicator of a periprosthetic joint infection, wherein the plural pieces of training data include at least one combination of data indicating a high probability of a periprosthetic joint infection;
B) using the plural pieces of training data to pre-train a machine learning model;
C) wherein the plural pieces of training data of B) correspond to a combination of data that is associated with periprosthetic joint infection;
D) feeding plural pieces of data obtained from a subject to the machine learning model;
E) determining the likelihood of a subject having a periprosthetic joint infection based on an output of the machine learning model; and
F) treating the periprosthetic joint infection.

14. The method of claim 13, wherein treating the periprosthetic joint infection comprises treating the subject with an antibiotic.

15. The method of claim 13, wherein, the plurality of data of A) and D) includes at least two of a test measuring protein concentration and/or total protein content, a test measuring red blood cell concentration, a test measuring white blood cell concentration, a neutrophil or polymorphonuclear cell percentage, a test measuring c-reactive protein concentration, a test measuring alpha defensin concentration, a test to detect presence of microbial antigen, a test measuring calprotectin, a test measuring neutrophil elastase, a test measuring leukocyte esterase, a test measuring lipocalcin, a test measuring monocyte-to-lymphocyte ratio, a test measuring neutrophil-to-lymphocyte ratio, a test measuring platelet-to-lymphocyte ratio, a test measuring absolute neutrophil count, a test measuring d-dimer, a test measuring erythrocyte sedimentation rate, a test measuring lactate or L-lactate, the affected joint, a subject's age, and a subject's gender.

16. The method of claim 13, wherein the plurality of data comprises a panel of information that is fed into the machine learning model.

17. The method of claim 13, wherein the machine learning model comprises at least one of a logistic regression model, a support vector machine model, a decision trees model, a random forests model, an adaptive boosting trees model, a gradient boosting trees model, an explainable boosting machine model, a nearest neighbors model, a neural networks model, a KMeans model, a gaussian mixture model, a hierarchical clustering model, a density-based spatial clustering of applications with noise (DBSCAN) model, a fuzzy clustering model, a principal component analysis (PCA) model, a linear discriminant analysis (LDA) model, a factor analysis of mixed data or factorial analysis of mixed data (FAMD) model, a single value decomposition (SVD) model, and a t-distributed stochastic neighbor embedding (t-SNE) model.

18. The method of claim 13, wherein the machine learning model is a first machine learning model and the method further comprises subjecting the plurality of data to a second machine learning model that is different from the first machine learning model.

19. The method of claim 13, wherein the output of the machine learning tool is compared against a threshold value, the output of the machine learning tool is delivered as a probability score, the output of the machine learning tool is delivered as a rank-percentile, the output of the machine learning tool is delivered as a confidence score, the output of the machine learning tool is delivered as a categorical range, or a combination thereof.

20. The method of claim 19, further comprising G) determining the threshold value.
